(19) United States
(12) Patent Application Publication
(10) Pub. No.: US 2019/0197055 A1
(43) Pub. Date: Jun. 27, 2019

(54) HEAD MOUNTED DISPLAY USED TO ELECTRONICALLY DOCUMENT PATIENT INFORMATION AND CHART PATIENT CARE

(71) Applicants: Rafael A. RODRIGUEZ, New York, NY (US); Sharif PROFIT, Bronx, NY (US)

(72) Inventors: Rafael A. RODRIGUEZ, New York, NY (US); Sharif PROFIT, Bronx, NY (US)

(21) Appl. No.: 16/286,052

(22) Filed: Feb. 26, 2019

Related U.S. Application Data
(63) Continuation of application No. 15/237,980, filed on Aug. 16, 2016, now abandoned.
Application No. 15/237,980 is a continuation-in-part of application No. 14/757,756, filed on Dec. 23, 2015, now abandoned.
(60) Provisional application No. 62/096,410, filed on Dec. 23, 2014.

(51) Int. Cl. G06F 16/70; G16H 40/20; G16H 10/60

(52) U.S. Cl. CPC G06F 16/70; G16H 10/60; G16H 40/20

(57) ABSTRACT

A head mounted display, typically in the form of glasses or eyewear, can be used to electronically document patient information and chart patient care. The head mounted display device can interface with EMR software to allow a physician to do patient care tasks and rounds, simultaneously documenting in the patient record by giving verbal commands to the head mounted display. The patient chart and history can be made visible to the physician through the eyewear, allowing the physician to move freely from patient to patient. The eyewear display can also be able to interface with other medical equipment to pull in vital information from heart monitors, respiratory devices, IV devices, and other numerical valuations involved with patient care. As a result, the physician can provide superior patient care without losing precious time spent sitting at a computer for performing documentation and charting.

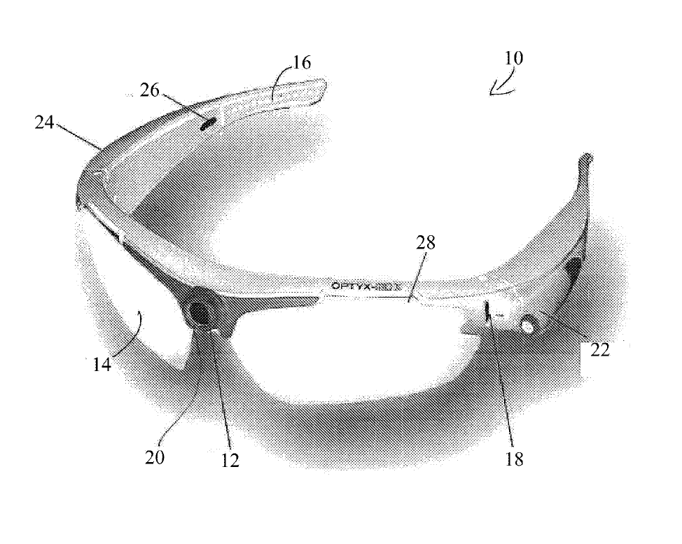

### CROSS REFERENCE TO RELATED APPLICATIONS

This application in a continuation-in-part of U.S. patent application Ser. No. 14/757,756, filed Dec. 23, 2015, which claims the benefit of U.S. provisional patent application No. 62/096,410, filed Dec. 23, 2014, the contents of which are herein incorporated by reference.

### BACKGROUND OF THE INVENTION

The present invention relates to electronic medical record systems and accessories and more particularly, to a head mounted display/eyewear that can be used to electronically document patient information and chart patient care.

Due to the mandated implementation of electronic medical records (EMR's) in hospitals and healthcare systems, the patient care is dynamically affected. The amount of time spent documenting doctor's orders and patient care in computer software systems becomes time consuming and limiting to patient care. Physicians and nurses are complaining of spending more time on the computer than with their patients.

Current EMR software programs require doctors to be at a computer entering orders manually and searching through a database of orders and order sets. This conventional process is cumbersome and patient charts can be dense, requiring the physician to click through page after page of several screens, clicking on multiple choice items or drop down menus. Moreover, navigating these conventional systems can be time-consuming and difficult. The added time required for physicians to work with these systems often is taken away from patient care and focus.

As can be seen, there is a need for a system that can satisfy a physician's and nurse's use of EMR's while requiring minimal time to review, enter and manipulate data.

### SUMMARY OF THE INVENTION

In one aspect of the present invention, a method for providing and gathering information to and from medical personnel comprises disposing glasses on the medical personnel, the glasses including lenses for displaying information and a wireless communication system for sending and receiving information.

In another aspect of the present invention, an computer implemented method for providing patient care comprises wearing, by medical personnel, glasses having a wireless connection configured to interface with an electronic medical record of a patient; reading, by a camera disposed on the glasses, a patient identifier, the patient identifier identifying the patient being seen by the medical personnel; and receiving, via the wireless connection, a medical record associated with the patient, the medical record being displayed to the medical personnel on the lenses of the glasses

These and other features, aspects and advantages of the present invention will become better understood with reference to the following drawings, description and claims.

### BRIEF DESCRIPTION OF THE DRAWINGS

The FIGURE is a perspective view of eyewear forming a head-mounted display, according to an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a head mounted display, typically in the form of glasses or eyewear, which can be used to electronically document patient information and chart patient care. The head mounted display device can interface with EMR software to allow a physician to do patient care tasks and rounds, simultaneously documenting in the patient record by giving verbal commands to the head mounted display. The patient chart and history can be made visible to the physician through the eyewear, allowing the physician to move freely from patient to patient. The eyewear display can also be able to interface with other medical equipment to pull in vital information from heart monitors, respiratory devices, IV devices, and other numerical valuations involved with patient care. As a result, the physician can provide superior patient care without losing precious time spent sitting at a computer for performing documentation and charting.

The device of the present invention allows physicians to better manage their time doing computer physician order entry simultaneously when visiting their patients, not while sitting at a computer trying to navigate EMR software. The physician can speak orders and patient care tasks into their eyewear and those orders and tasks can be documented wirelessly into the EMR and patient charting software.

The device of the present invention can integrate with all systems, including but not limited to Windows, IOS, Mac, OS, Android, Linux, and the like. Moreover, the device of the present invention can be used to do patient charting on all EMR/EHR systems, including but not limited to Epic, Cerner, Allscripts, Eclypsis, Soarian, Meditech, McKesson, and MedHost.

The device of the present invention can be used by all staff at hospitals, including patient care staff, nursing staff, physicians, specialists, physician assistants and the like. Appropriate protocols can be implemented to allow certain personnel to have access to certain data, including read and write privileges. These protocols can also help ensure privacy of the patient as well as HIPPA compliance. Security measures to ensure that appropriate personnel can access the device include voice recognition, retina scanning, password entry, and the like.

Referring now to the Figures, in a hardware aspect of the present invention, a device 10 (also referred to as glasses 10) can be a HMD (Head Mounted Display), HUD (Head Up Display), or AR (Augmented Reality) Smartglasss, as well as all variations of a high tech pair of wearable technology, thin client, directed via voice and eyes. The device 10 can include a video camera 12, video screen 14 (also referred to as lens 14), video projection, speaker 16, microphone 18, touch pad (not shown), and motion sensor 20, that interface with current and future hospital technologies and third party software and hardware. The device 10 can be used in multiple roles and multiple healthcare settings creating, for example, an intraweb to connect to a larger medical/hospital network. Like every hospital device, the device 10 of the present invention can be tested and designed to meet HIPPA regulations and hardware safety and sterilization needs. To ensure the protection of data sensitive information, the device 10 can provide biometrics, such as retina scanning and other such methods, to ensure the security appropriate to the user.

The convergence of hardware and software, combined with wearable sensor technology, provides opportunities for hospitals to streamline their use of Electronic Records and provides innovation in the healthcare marketplace and the practice of medicine. The user experience becomes fundamentally different from current mobile technology in a clinical setting, such as desktops, tablets and smartphones. As a wearable device that is always on and ready, it has remarkably streamlined clinical workflows that involve information gathering and information sharing. The glasses 10 are handsfree and non-immersive; they encourage the user to be present in their interactions with the patients while seeing their own surrounding enhanced with the data and information they need to enhance patient treatment and patient care. The device 10 also enables the continued fluidity of the medical practice to continue. The device 10 of the present invention allows physicians to better manage their time doing computer physician order entry simultaneously when visiting their patients, not while sitting at a computer trying to navigate EMR software.

The device 10 offers various functionalities, including patient identification and facial recognition, simple video streaming of procedures and recording patient care activities, information sharing and video/image projecting, to overlaying x-rays, cts and other imaging for providers during surgery, to easily accessing patient information, records and charts while performing day to day duties for all hospital staff such as bedside medication verification, accessing patient health information, and overlaying information at the point of care. The device 10 also offers health applications, providing magnification of images, lights, stethoscope capabilities, laser mapping and stability grids and the ability other medical handheld devices provide and an interface to connect with other important devices such as chemo treatment machines.

Examples include interfacing patient monitoring devices directly to the device 10, receiving real time alerts of results for labs and allowing providers to use natural dictation devices, such as dragon, to dictate their charts as they move around the hospital. Nurses and physicians will read/write via voice commands to any EHR/EMR system. Surgeons will be able to view x-rays, labs, and the like, while in an operating room without leaving to scrub out and back into surgery. Surgeons can be provided with special MDX which can be sterilized after every use.

The device 10 can become integrated in current medical workflows. For example, when a clinician walks into department room, he or she will have an identification trigger, such as a QR code, on the wall or a patient barcode sticker. The device 10 can immediately recognize the room and then the department specific electronic health record sends information about the patient that is in that room to a menu displayed on the device 10. The clinician can speak with the patient, examine the patient, and perform procedures while still seeing problems, vital signs, lab results and other data in view on the screen 14 of the glasses 10, prompted by voice commands and assisted gadgets. A provider could use the glasses 10 to look at a monitor, and another provider could have a tablet down the hall and could see exactly what they're seeing. Enabling the movement of providers and clinicians is vital to proper patient care, which has suffered with current technologies, such as desktops. The technology of the present invention can provide the ability to stream live images of a patient's medical condition to a consulting specialist located elsewhere. For example, an ambulance crew responding to a stroke victim can use the glasses 10 of the present invention to provide real-time video and audio to a neurologist back at the hospital who could then order a clot-busting, brain-saving drug immediately.

The non-immersive glasses 10 can encourage the user to partially see their own surrounding using a live view screen liquid crystal display (LCD) or organic light emitting diodes (OLEDs), for example. The lens in the glasses can amplify the graphic, giving the end use the illusion of a greater-sized screen. The lens in the glasses may provide not only a two-dimensional display of information but may also provide a display in three-dimensions, allowing a user to move information to the background or foreground as desired, without losing that information on the screen. A three-dimensional display may also be projected, as discussed below, to a surface or in space as a hologram, to allow a user to display to another person a three-dimensional device, such as a human organ, for example.

The device 10 can be equipped with a microphone 18 for audio recognition of spoken word commands. Ear buds (not shown) can be optionally added for an in-ear speaker. The ear buds can be wired to the device, or can receive sounds wirelessly from the device. In some embodiments, the microphone 18 can be actively monitoring the physician's voice for specific commands. In some embodiments, an on/off button can be provided so that the microphone 18 does not pick up inadvertent information.

The device 10 can include a light 22, such as an LED light, to allow the physician to perform illuminated patient examination. The light 22 can be powered on and off as desired.

The device 10 can include a wireless interface protocol (generally shown at 24) for communicating with other devices or with an EMR software or patient charting software. The wireless interface protocol could include Bluetooth, Wi-Fi, or the like.

The device 10 may communicate with other devices, such as patient vital sign monitoring devices. In this embodiment, a vital sign monitoring device may send information to the device 10 to be displayed thereupon. The vital sign monitoring device may be a machine in a patient's room, such as a pulse oximeter, for example, or may be a device in a surgical suite, allowing a surgeon to “see” the vital signs without having to turn one's head to look at a machine.

The device 10 may also receive signals, such as doctor pages, and display the pertinent information on its screen. Thus, instead of having to page a doctor (electronically, or via a voice page throughout a facility), the device 10 can receive messages and display them on a screen. Moreover, a doctor wearing the device 10 can be alerted of a patient's vital signs when away from the patient, thereby alerting a doctor (or other such professional) before a patent “crashes”. In conventional methods, a patient may, for example, experience a cardiac arrest event, the vital sign monitoring machines detect such event and alert a “code”, where the medical professionals then respond. With the present invention, a medical professional wearing the device 10 may be alerted of adverse vital signs prior to the “code” or may be immediately alerted of a “code” without having to be paged.

The camera 12 can be provided in the device 10 for recording pictures or videos, as desired. The pictures and/or video can be uploaded from the device 10 to the patient's chart or the EMR system as desired.

The device 10 can be powered by various means, including a rechargeable and/or replaceable battery source. A charging port 26 can be provided to deliver electric energy to the device to recharge the batteries. The charging port can be, for example, a micro-USB port.

The lenses 14 of the device 10 can take various forms. For example, they may be clear lenses that can be used as a display from an inner surface thereof. The lenses 14 can include other features, such as a flip-up transition lens 28, depending upon the specific application.

The device 10 may further include a mechanism to send a portion of the display onto another surface. For example, this mechanism may include a projector that allows the physician to, for example, remove the device 10, place the device on a surface, and transmit date displayed on the device to, for example, a wall of an examination room. This may be useful to show the patient results from tests or the like. In addition to projection, all or a portion of a screen may be sent to a monitor or other type of display interface via a wireless protocol, such as Bluetooth, Wi-Fi, near field communication (NFC) or the like. Similarly, the device 10 may be used to project a hologram, such as a three-dimensional display of a procedure to be performed, an anatomical display, or the like.

The device 10 of the present invention can be used not only in the medical community, but in other work environments that require documenting details and work flow process. These areas can include, for example, construction, agriculture, and the like.

In some embodiments, the device 10 can include a live translation feature, where, when activated, a translation of what is being said to the user may appear on the lenses 14 of the device 10. In this embodiment, the microphone may receive the words being voiced to the user. In some embodiments, a translation software may receive the voice, optionally convert the voice to text, translate the text to a desired language, and send that translated text back to the lens 14 for reading by the user. In other embodiments, this feature may be achieved by sending the voice to a live translator who may type in the direct translation that would then appear on the lens 14 of the device 10. The translator may be at the particular facility or at a remote location. In some embodiments, software may be used to determine the language and then the voice may be sent to the appropriate translator. This feature may be activated in various manners, such as via a voice command, by a particular swipe or menu option on the lens 14, by a hand gesture, or the like.

It should be understood that operation of the device 10 can use various mechanisms. Eye movements, hand gestures, physical touch, voice commands, mental thought processes, or the like, may be used to enter various modes or commands into the device 10.

Various workflows can be contemplated by the device 10 of the present invention. Below are several such workflows set forth as examples, without limiting the scope or spirit of the present invention.
Admission—From the Emergency Department (ED)
Before the patient leaves the ED:
   1. “ED intent to admit” order is entered in Cerner (or other such similar system); order details including reason for admit, patient status, and nurse unit type.
   2. “ED intent to admit” icon appears on the tracking board.
   3. Request for a bed is placed with admitting or the house supervisor per facility process.
   4. ED physician consults with admitting physician and may place these orders as well if he/she is available.
      The patient will get a bed if the ED transition order does not have a patient status.
      If any orders are to be continued from the ED, right click the order and change the ordering physician to the admitting physician's name.
   5. Admitting receives a requisition and bed assignment is provided by Admitting/Bed Control/House Supervisor as per facility process.
   6. Bed number is entered as a comment on the ED Tracking Board.
   7. ED nurse gives report to receiving nurse (see Patient Handoff).
   8. ED documentation is completed (nurse and physician).
   9. Patient is transported to the assigned bed.
   10. Once the patient is physically out of the ED, the ED nurse accesses the Depart Process and indicates that the patient was admitted and addresses notifications of any incomplete tasks.
      When the patient arrives on the nursing unit:
Bedside Monitoring Device Interface (BMDI): For patients in Intensive Care and monitored areas, recovery areas, and emergency departments, it is not necessary to use a Bedside Monitoring Device Interface (BMDI) to document vital signs and ventilators.
Patients must be associated to the monitor on admission to a unit and disassociated from the monitor on discharge from the previous unit. To complete admission history:
   1. A task appears on the task list to complete Admission History.
   2. Nurse clicks on the task to access the PowerForm (within 24 hours of hospital admission) for Admission Assessment, which will be tasked as done/not done. Nurse completes and documents admission assessment in IView (within 8 hours of admission in a non-critical care area and within 1 hours in a critical care area).

Interdisciplinary Plans of Care (IPOCs) may be suggested based on nursing documentation. The nurse should evaluate the appropriateness of suggested IPOCS for the patient and initiate those that are appropriate. When initiating an IPOC, the nurse will select goals indicators, and interventions specific for the patient's condition and needs.

If the nurse, based on clinical judgment, determines that an IPOC which has not been suggested is appropriate for the patient, the nurse may search for and initiate additional IPOCs by selecting Orders/Add from the menu on the left of the screen and searching IPOC. The nurse initiates the IPOC within the 24 hours of admission based on information obtained in the admission history and assessment.
3. The nurse reviews and completes orders and tasks as described in Organizing the IP Day.

### Direct/Post-Op

When the patient arrives on the nursing unit:
   1. Admitting assigns a patient to a room when the patient is in the OR/PACU and notifies the Charge Nurse of the need for a bed.
   2. Center Staff Assignment is updated prior to the patient arriving on the floor and Charge Nurse gives Admitting the room number. Admitting updates STAR with the assigned patient location.
   3. A task appears on the task list to complete Admission History.
   4. Nurse clicks on the task to access the Admission Assessment PowerForm, which will be tasked as “done/not done.” Nurse completes and documents admission assessment in IView (within 8 hours of admission in a non-critical care area within 2 hours in a critical care area).

If the patient is a post-op-admit, some admission data collected pre-operatively will pull forward into the Admission History and Assessment PowerForms.

Admission history data should be reviewed and updated as appropriate. Consistent documentation of “no PMH” can be completed by using the No Chronic Problems button located on the Handoff screens, Inpatient Summary, and Problem List.
5. Nurse initiates the Interdisciplinary Plan of Care (IPOC) within 24 hours of admission based on information obtained in the admission history and assessment.

### Organizing the IP Day

1. Off going Charge Nurse creates assignments on paper. Assignments are transcribed into PowerChart.
2. Charge Nurse or designee completes staff assignment in Cerner to match PowerChart.
3. Charge Nurse is notified of any discrepancies and correct them.
4. Either the Nursing Assistant or the nurse (as ordered) takes and documents vital signs using CareMobile or into IView under the Adult Quick View band.
5. Assessment is completed in iView. Use any additional. bands as indicated by the patient's condition.
6. Begin assessment and document them in the system.
7. Document IV/Central line site assessments or interventions in the Adult Lines-Drains IView band.
8. Monitor CareCompass/task list of tasks that are due throughout the day and address the tasks. Remember to mark “done/not done” to get rid of the task.
9. Monitor CareCompass/task list for new orders and review any orders flagged for nurse review.
   Orders entered by a nurse are not flagged for nurse review. If the physician enters orders, the nurse will need to complete a nurse review. If orders are entered as written orders from a paper order, they will need to be validated against the paper order and signed-off on the paper chart by the nurse.
   Order Entry Details PowerForms will be tasked and must be completed at 2 am.
   PowerForms will not be tasked must be updated with every unit-to-unit transfer.
10. Throughout the day, administer medications as they are due (or indicated on a PRN basis) using either CareAdmin (PowerChart—medication administration) or CareMobile (hand-held device) to document on the eMAR.
11. Document pain assessments at least every 4 hours (may be documented in Adult Quick View IView band or Pain Assessment PowerForm).
   Reassessment following medication interventions for pain will be tasked and should be documented within 1 hour of the intervention. Document reassessment by clicking the task from the MAR. Both of these options open the Medication Response Follow-Up PowerForm.
12. Document any physician notifications (including critical test results) in the Provider Notification PowerForm. If a notification is about a critical test result, be sure to indicate the result in the comment field and call the physician.
13. Document all patient education in one of the Education PowerForms or in the Education IView band.
14. Review and update IPOC based on patient's condition and responses to care (during every shift). IPOCS must be updated at least every 24 hours.
15. Document I&O in the Intake and Input IView band. This may be done as the I&O occurs throughout the day. The system will calculate totals.
   When clearing volumes infused from IV pumps, validate that the volumes cleared for the shift match the volumes recorded in the record.
16. Prepare for handoff to the next nurse by making sure that overdue tasks, medications and the IPOC(s) are addressed. This should include:
A final “chart check” of the paper chart near the end of each shift to ensure that any paper orders have been transcribed and entered into the Cerner system.
A “12-hour chart check” that tasked to the nurse at 0500 and 1700. This is a “done/not done” task and must be addressed.
A review of orders and tasks as described in the Organizing the Day on an IP Unit guide.
   Some forms will remain on paper (MetTeam, Code Sheets, Interfacility Transfers, etc.).

### Entering Allergies, Height & Weight

Nurses are required to complete allergies, height, and weight in the new Cerner system. Failure to do so can cause rework, delays in patient care, delays in getting a patient's medications filled, and other patient safety issues because Pharmacy cannot fill an order without allergy, height, and weight entered in the system. To complete allergy information (EXAMPLE):
1. Open PowerChart.
2. From the menu, select Allergies.
3. Click Add.
4. In search window, type “codeine.”
5. Click Search to left of window.
6. “Codeine” is displayed on right side of page under Substance.
7. Category displays drug or change if needed.
8. Complete “reaction type of allergy.”
9. Click Reaction Type and choose “rash.”
10. Complete status and select Active.
11. Enter severity as “low.”
12. Click Apply and then OK.

### IView: Document Results

1. Right-click the IView window and select “Insert Date/Time.” A column appears with the current time.
2. To set a different time, right-click the inserted column and select “Change Date/Time.”
3. Select the appropriate date and time.
4. Click outside of the date and time boxes to update.

### Medication Reconciliation

Physicians are solely responsible for medication reconciliation at admission, transfer, and discharge.
   Nurses do not have access to perform the medication reconciliation functionality in the Cerner system.
   All transfer, the Order Reconciliation function can be used by the physician to reconcile medications and all orders for care in the new venue (e.g., EI vitals, activity, labs, etc.)
   A nurse can take telephone orders for medication in order to provide care for the patient.
   Nurses will continue to be responsible for collecting Medication History (home medication list). See below.

### Medication History

Collection of the medication history is not medication reconciliation. To collect Medication History:
1. Open PowerChart.
2. From the task list (if tasked) or Ad Hoc Forms, select or open:
   General Nursing—Admission, Adult History
   General Nursing—Admission, Peds History
   Surgery/Procedure—PAT/Pre-Procedure (Adult, Outpatient/Pediatric)
   SNF—Select Orders from the Menu.
3. Scroll down to and click Medication List; the Orders page opens. If already using the Orders page to add home medications, skip this step.
4. Click Document Medications by History.
5. Click Add to begin the home medication list.
6. Complete all the information to the degree possible. Selecting the closest medication order sentence and modifying the details will save time.
   If there is a medication change per family or patient and medication history is updated, the nurse must call physician to inform of change.

### Form Browser

Use the Form Browser to view a list of forms completed for the patient during the specified time range.
   1. From the menu, open Form Browser.
   2. Double-click on a form to view the documentation. The completed form displayed is in read-only format.
   3. Right-click on the form to modify, print, or unchart a signed form.
      To unchart a signed form, right-click the form occurrence and select Unchart to open a comment dialog box.
      Enter an uncharting reason in the Comment box.
      Click the check mark to chart the information.
      The results displayed on the flowsheet for the patient will display as “in error” and in the Form Browser displays the form with a strike-through.
   4. Click the X in the upper-right to return to the previous window.

### View Results

1. From the Menu, open IView.
2. Right-click the result you need and select View Result Detail or double-click the cell.
3. To view the caregiver who entered the results, click Action List.
4. Click Close to exit from the result details.

### Order Management

Physician places and signs orders via Computerized Provider Order Entry (CPOE); the Cerner system automatically sends them to the appropriate department. Nurses are responsible for:
   Verifying what has been ordered
   Verifying they were correctly processed by the system.
   Ensuring that all orders are completed from the last shift or in a timely manner.
   Completing Nurse Review by clicking the Nurse Review button and addressing orders.

### Initiate a Planned PowerPlan

(Example: Orders started in the PACU. In this scenario, the Anesthesiologist placed a post-op PowerPlan on the patient with instructions to initiate the orders when the patient arrives on the inpatient unit.)
   1. Navigate to the Orders Profile.
   2. In the Plans—Medical section, PowerPlans for the patient will display. Locate the planned PowerPlan to Initiate. Plans that are active display in bold whereas plans that have not yet been initiated display in regular font.
   3. Select the planned PowerPlan and review the details as necessary. If there are any concerns about a particular order or orders, call the physician for clarification before initiating.
   4. Click the Initiate button.
   5. Click the Orders for Signature button.
   6. Click the Sign button.
      The nurse cannot remove any orders by unchecking. If an order is needed that exists in a plan, the nurse should contact the physician for an individual order. Orders that are signed but not initiated will remain in planned status until they are initiated. If they are in a planned state, note that the orders don't go to any other department. Orders that are signed and imitated are active orders.

### Choosing the Correct Communication Type:

When entering an order into Cerner, it is important to choose the correct communication type.
   Orders entered with a For Provider Co-Sign, Protocol, Telephone or Verbal communication type go to the physician for co-sign. The physician name entered into the order communication box determines which physician the order will be sent to for co-sign.
   When entering an order into Cerner, it is important that you select the correct physician who has placed the order. Do not enter the admitting physician by default. Pay careful attention to the full name of the physician and avoid entering in a different physician with the same last name.
   If you don't close the chart, the same physician's name appears as a default in the chart.
   Enter Paper Order communication type when transcribing a written order into the electronic record. All paper orders must be placed in the patient paper chart to be scanned into the medical records after discharge. Orders entered as a paper order communication type do not go to the physician for co-sign, as there is a corresponding, signed written order in the paper chart.
   Enter Per CPOE communication type if an order (such as a lab test) must be cancelled and reordered to change some of the order details. To complete this process, right click on the order in the order profile and select “Cancel/Reorder.” Use this process when you need to:
      Change the collection method for an existing lab order from “Nurse Collect” too “Lab Collect” or “Lab Collect” to “Nurse Collect” due to a change in the patient condition (such as removal or insertion of a central line) or location
      Change the appropriateness for room service information on an existing diet order due to a change in the patient's condition (e.g., a cancel reorder would be completed when you do Order Entry Details)

### Medication Administration

Nurses in inpatient units must complete Nurse Review and ensure Pharmacy has verified all medications before administering medications. Once this has been done, complete the following steps to administer medications:
   1. In the med room, with the patient's eMAR open on the computer screen, identify medication(s) to be administered.
   2. Retrieve medication from the Medication Dispensing Cabinet or other storage area in the med room verifying the 5 rights against the eMAR.
      Do not remove medications from their original wrappers in the med room. The bar code will be needed at the bedside. If a medication needs to be crushed or split for administration, obtain a single patient use crusher/splitter and crush/split the medication at the bedside.
      For a select member of medications a unit dose may not be available. Patient specific labels are to be supplied by the Pharmacy for these.
   3. Take the medication(s) to the patient room.
   4. If using CareMobile, log into the CareMobile device:
      Scan the barcode on the patient's wristband.
      Click the Medication Administration button on the toolbar.
      Select Scheduled Medications folder from the drop down menu.
      Navigate to the appropriate medication folder (scheduled, prn, etc.).
      Scan the barcode for each medication. If the medication exactly matches the medication ordered (as displayed on the MAR), you will see a check box. If additional information is required for the medication, chart any additional details or comments required.
      Tap the Save button.
      Administer all medications.
      When all medications have been entered, tap the Signed drop-down menu and go to the To Be Signed folder.
      Review the items listed for signature and sign your documentation.
         Patient refuses the medication or is unavailable when a scheduled medication is due?
   Scan the medication and document as “Not Given,” providing the reason (e.g., patient unavailable or patent refused).
      Can't scan the medication?
   If a medication cannot be scanned, document it manually on the eMAR.
   1. Right click on the medication from the eMAR and select Med Request.
   2. Type in the comment that the “barcode would not scan.”
   3. Administer the medication manually without scanning from CareMobile.
      When possible, retain the medication wrapper with the barcode and return it to Pharmacy for troubleshooting. A place will be designated for medication wrappers in all nursing units.
      Remember to pay close attention to any warning messages when scanning medications. If you receive a warning, recheck the 5 rights and review the order for the medication. If all the information is correct, (patient, drug, dose, route, strength, and timing), contact the Pharmacy for verification and then administer the medication.

### Administer Medications Using CareAdm in

The physician has ordered a medication. One needs to administer the medication. From the patient's chart:
   1. Verify medication ordered in eMAR.
   2. Click the Medication Administration button in the toolbar.
   3. Scan the patient's wristband.
   4. If a wristband has been damaged, print a new one.
   5. The timeframe can be changed to view additional medications.
   6. If you administer the medication late, you are prompted to indicate a reason for the delay.
   7. Verbally confirm the patient identifiers.
   8. Scan the medication.
   9. The result column indicates current amount scanned.
      eMAR: Change Timeframes
   1. After scanning patient's wristband, right-click the Timeframe bar in the Medication Administration Wizard, and select “Change Search Criteria.”
   2. The Search Criteria dialog box opens.
   3. Modify the Form and To date and times to the appropriate time period.
   4. Click OK.

### Patient Handoff (Shift & Unit-to-Unit)

Patient handoff is completed while nurses review a summary of patient care together in the CareCompass, Patient Handoff, and Plan of Care Summary sections of the EHR. For shift to shift, both nurses should be at the bedside. For unit-to-to unit, the report may be given over the phone with both nurses looking at the patient's chart.
Complete the following tasks for every patient handoff:
   1. Review and resolve any overdue tasks.
   2. Review and resolve medications that are overdue or due soon.
   3. Review all orders and note any orders in a planned state that need to be activated by the receiving nurse.
   4. Review the Plan of Care and note the last time each IPOC was updated.
   5. Document the Hand Off Report in the Clinician Communication section of the HER.
   6. Review the eMAR.
      Sending nurse discontinues orders from their unit before sending patient to the floor. Receiving nurse activates orders for their unit when the patient arrives on the patient floor.

### Blood Products

A patient needing Type and Cross for surgery should complete Preadmission Testing (PAT) at the facility where the surgery will be performed. It is important to note the following:
Each facility will have a policy about placing a blood band on the patient.
The consent for a patient to receive blood products is covered in the surgical consent. No additional consent is needed.
Intraoperative nurses do not need to place an electronic order for blood products. Continue to follow the current practice to obtain and pick up blood products. (Blood Bank will enter any necessary orders.)
No orders is needed to return blood to the Blood Bank.

### Blood Administration

The MED Blood Transfusions KY PowerPlan will be used to order blood products. If the blood products is not ordered to be transfused at the time the PowerPlan is signed, and the physician decides the product should be transfused, update the original MED Blood Transfusion KY PowerPlan by selecting and signing the appropriate Transfuse order.

Once blood is ready for pick up, the nurse will be tasked to pick up in the Blood Bank. The Blood Bank will not be calling the floors.
Blood Administration completed by nursing will be documented in the Blood Administration IView band.
Complete the Blood Administration Initiation Information at the bedside while two nurses are present.
When the administering nurse clicks the sign button, a box will appear for the verifying nurse to “sign.”
Verifying nurse enters their login information as their electronic signature. This will drop a charge as blood products aren't scanned.
When documenting initiation, enter the blood unit ID numbers into the Blood Unit ID Number field.
Per policy, a patient assessment must be performed and documented during the transfusion. This should be documented in the Patient Response field in IView.

### Document Blood Administration IView

Once 1) the blood products is ready for your patient, 2) proper patient identifiers for the patient have been confirmed, 3) the user and the witnessing nurse have verified the blood product and patient information, document blood administration in IView by following these (steps below are for documenting packed red blood cells):
1. Click IView/I&O from the Menu.
2. Scroll down to and click on the Blood Administration band.
3. Scroll down t and click on the Blood Administration Initiation band.
4. Select the graph icon to create a Dynamic Group using an appropriate type of blood Unit ID, Unit Type, Product.
5. Click Dynamic Group icon.
6. Enter Blood Unit ID Number for each unit. The time can be changed by right-clicking the time field and choosing “Insert Date and Time.”
7. Click Sign.
   When the blood has been infused in the OR, document the total volume infused from all units in the Blood Product Intake band (directly beneath the Blood Administration Initiation band). If the unit is still infusing when departing from the OR, the OR nurse should inform the PACU/Unit nurse of the total infused in the OR. The PACU/Unit nurse will record the total in IView when the unit has infused. Document this report in the Departure from OR segment.
      STEPS 1-6 should be repeated for each unit of blood transfused. Document other blood products by completing steps 1 and 2 above; click the Blood Product Intake band and enter the amount infused in the appropriate field.

### Depart Process (Discharge)

1. Physician enters the discharge PowerPlan in Cerner, completes discharge medication reconciliation, enters the discharge diagnosis, enters follow-up information, and dictates or users electronic documentation for the discharge summary. Physicians can use DynaDoc PowerNote or traditional dictation for discharge notes.
2. Nurse accesses discharge documentation through the Discharge Summary in the menu.
3. The list of items displayed is “depart actions.” Actions in yellow are required and should be completed prior to discharging.
4. Nurse completes the following additional steps:
   Add any additional follow-up information not already entered by the physician.
   Select medication leaflets and patient education documents to provide the patient with the discharge paperwork.
   Complete and sign the Discharge Summary and Discharge Instructions paperwork. This information can be saved by clicking “Save Draft” or “Print and Save.”
   When all information is complete, print patient information by clicking the “Sign and Print” button at the bottom-right of the depart process window.
   Provide all prescriptions and other instructions to the patient. The patient should sign the printed patient discharge sheet and a copy should be placed in the paper chart (print two copies). Continue to fax information to physician offices until further notification. This should be noted on the copy of the discharge sheet in the paper chart.

### Transfer to Another Facility

This is the same as the discharge process with these additions:
   1. The indicated chart documents are provided for the receiving facility.
   2. If documents are electronic, they are printed.
   3. If documents are part of the paper record, they are copied.
   4. Report is called to the receiving facility.
   5. Transport to the receiving facility is arranged.

### Disposition of Body

1. The Expiration Record PowerForm is completed at the time of patient expiration.
2. The Disposition of Body section of this form should not be completed until the body has been released to the appropriate entity.
3. When the body is released to the appropriate entity, staff modifies the Expiration Record PowerForm and completes the Disposition of Body section.

### Specimen Collection

When there is an order for a specimen that is to be collected by the nurse, a task will appear on the task list both in PowerChart and on the handheld CareMobile device.
Take the printer, supplies needed to collect the specimen, and the handheld CareMobile device to the patient's bedside.
Print the specimen label from either PowerChart or the handheld CareMobile device:
   From PowerChart, right click on the task and select Print Label. The label will print to selected printer.
   From the CareMobile device:
      Hold the stylus down on the lab item for which you need the label.
      Select Print Label.
      Scan the desired printer.

### Collection of Specimen:

1. Log onto CareMobile.
2. Scan the patient's ID band (this will pull up the patient information on the device).
3. Navigate to the Specimen Collection folder.
4. Draw patient specimen.
5. Select the specimen task that is collect.
6. Scan the specimen label.
7. Sign the specimen collection form in CareMobile.
8. Task is complete and the order goes to a “collected” status.
   All labels will be printed by the clinician at bedside except for Jewish downtown ED, MCE ED and MCS ED where labels will automatically print to a designed printer.

### Labeling the Specimen at the Bedside:

If CareMobile is not used in the specimen collection process, you must write your initials, date, and time on the specimen label. For blood cultures, the specimen collection site must be written on the label. When writing on the label, do not write on the barcode as this can interfere with scanning of the barcode in the lab.
Line, RT, Lab will be called out. Circle so Lab to know who gets the charge. See below.

### Preop Workflow

### Patient Identification and Validation

1. Verify the patient's FIN# on the wristband matches the FIN# in the Case Selection and the Banner Bar in the patient's chart. If there are any discrepancies, STOP documentation, so not initiate any orders, and notify the Charge Nurse.
2. Verify the procedure with the patient (Schedule, H&P, and consent). If there are any discrepancies (procedure and/or laterality), STOP documentation and notify the Charge Nurse. (The surgery may need to be rescheduled in the scheduling appointment book so the information on the Tracking Board and the medical record are correct.)

### Document Arrival Time

Document arrival of the patient via the Perioperative Tracking Board by setting a manual event. Unit Secretaries may also complete this task.

### Review Orders

1. From PowerOrders, review any PowerPlans or orders that may have been placed in a planned state by anesthesiologist or surgeon/physician.
2. Initiate if applicable.
3. If no orders are present, check for paper orders, then notify the Charge Nurse and contact the physician for orders if needed. All paper orders must be entered into Cerner as “paper” with the date, time, and authentication.
4. If order states to follow Anesthesia Guidelines, review the paper Guidelines and enter each order as a single. order. Place a copy of the guidelines in the chart and mark it for physician signature.
5. The ANES Day of Surgery PowerPlan should be ordered in a planned state by anesthesia.
6. After anesthesia evaluation, medications will be entered and initiated by the physician in the ANES Preop Powerplan.
7. Complete Nurse Review when the eyeglass icon appears in the Orders section. This is an ongoing process as new orders are entered.

### PowerForms

1. Select appropriate Preprocedure form in the Adhoc folder.
2. Complete all red asterisks and information pertinent to your patient.
3. Document areas specific to Core Measures and Meaningful Use. Refer to the Handout for Preferred Locations.
4. Select any other PowerForms in Adhoc folder as needed (e.g., Central Line Checklist, Procedural Documentation for A-Lines, Central Lines. Peripheral Nerve Block etc.).
5. Signed PowerForms are located in Form Browser for modifications if needed.
   Document IV Insertion in IView (includes Central and A-Lines)
1. In IView, select the Perioperative Lines.
2. Add a Dynamic Group to document the insertion.

### Specimen Collection (if Application to Facility)

Specimen will be available for collection after placing the order. If you DO NOT collect specimen, document it in the order as “Lab by Collect” by selecting “No” next to Nurse Collect. Labels should print after signing order to default printer in the unit. The order will be sent to Lab to obtain the specimen or available in Specimen Collect for the nurse.

### If the Nurse Collects the Sspecimen:

1. Select Specimen Collection from toolbar.
2. Scan the patient's ID band (Patient information and orders will display).
3. To print additional labels (if not printed when order was placed) select Label Reprint from the toolbar. Also follow this step if specimen is shown as “collected.”
4. Scan label on the specimen. A checkmark will appear next to the specimen name on the screen.
5. Sign the collection after all specimens have been collected. Write initials and time collected on specimen.
6. Send to Lab.

### Medication Administration

1. Review PowerOrders/MAR for medication orders.
2. Complete Nurse Review if the eyeglass icon is present.
3. Document administration of medications by selecting Medication Administration at the top of the screen and following prompts. If medications do not appear, change the search criteria from the date on the grey band.
4. Always scan. Report scanning issues with either Care Administration functions to Charge Nurse and IT.

### Perioperative Tracking Board

Keep the Tracking Board updated with manual events throughout the Preop process.

### Patient Pharmacy

Prescriptions will be sent to the patient's pharmacy electronically.
1. Select Patient Pharmacy from the toolbar to document preferred patient's pharmacy.
2. If late discharge is expected, ask patient if it is a 24-hour pharmacy to prevent transferring and cancelling prescriptions.
3. Remember to document if “no pharmacy” is available.

### Perioperative Documentation

The Case Times segment will display the “time in” from the manual event set on the Tracking Board when the patient arrived.

### Document the Remaining Times as Appropriate:

1. From Perioperative documentation, select the Pick List tab.
2. Document the type of patient care delivered (e.g., Preop Care Surgery for surgery/procedure patients of Preop Care Diagnostic IVR for radiology patients).
3. Document items used or if no items are used.
4. Click the disc icon to save.

### Handoff

Document the report handoff to intraop in the Preprocedure form found in the Preprocedure Checklist.
If the Checklist was signed of the patient is an Inpatient, the Preprocedure Checklist will be in Form Browser and you will need to modify it to update patient information.

### Finalize Chart

After documenting all case times and the supplies used in the Pick List, finalize chart by clicking the green flag. This allows for the charges to process.

### Preop Procedural Documentation

Complete procedure documentation for cases performed in preop area in the Intraop segments of Perioperative Documentation.
1. Select the Perioperative Documentation Intraop tab and complete documentation in the following segments:
   Case Times
   Case Attendees
   General Case Data
   Timeout
   Surgical
   Procedure
2. Select Case Logging Document from toolbar. Click to remove all sections except Timeout Document Reason.
3. Click OK.
4. Document in each of the above sections paying close attention to the number of pages in each segment. Every grey box must have a check mark.
5. After the five segments are complete, a green checkmark will display.
6. Complete the Pick List as the “no items used” for this Intraop section.
7. Save and finalize the Intraop document.
8. If applicable, add Med Administration, Equipment Fire Risk, or Departure from OR.
   Peripheral Nerve Blocks are not part of the Intraop documentation. These blocks are documented in Adhoc PowerForm.

### Preadmission Testing

### Validate and Identify the Patient

1. Verify the patient's FIN# on the wrist band matches the FIN# in the Case Selection and the Banner Bar in the patient's chart. If there are any discrepancies, STOP documentation and notify the Charge Nurse.
2. Verify the procedure with the patient (Schedule, H&P, and consent). If there are any discrepancies (procedure and/or laterality), STOP documentation and notify the Charge Nurse.

### Review Orders

Orders for PAT at this time will be paper orders which may be with the patient, faxed, or in the Document/Reports. All paper orders must be entered into Cerner as “paper” with the date, time, and authentication.
1. If order states to follow Anesthesia Guidelines, review the paper guidelines and enter each order as a single order.
2. Place a copy of the guidelines in the chart and mark it for physical signature.
   If you use the ANES PASS PowerPlan, the order must be placed and initiated by the PAT nurse.
3. Complete Nurse Review when the eyeglass icon appears in the Orders section. This is an ongoing process as new orders are entered.

### PowerForms

1. Select appropriate PAT PowerForm in the Adhoc folder.
2. Complete all red asterisks including Health History, Allergies, Medication History, Advance Directives, Social Habits, VTE Risk Assessment, and Vital Measurements (including BP, H&W, BMI).
3. Continue to document information pertinent to your patient.
4. Modify signed PowerForms as needed from the Form Browser.

### Specimen Collection (if Application to Facility)

Specimen will be available for collection upon placing the order. If you DO NOT collect specimen, document it in the order as “Lab Collect.” The order will be sent to Lab to obtain the specimen.

### If the Nurse Collects the Specimen:

1. Select Specimen Collection from toolbar.
2. Scam the patient's ID band (this will pull up the patient information on the device).
3. To print additional labels (if not printed when order was placed), select Label Reprint from the toolbar.
4. Scan label on the specimen. A checkmark will appear next to the specimen name on the screen.
5. Sign the collection after all specimens have been collected. Write initials and time collected on specimen.
6. Send to Lab.

### Patient Pharmacy

Prescriptions will be sent to the patient's pharmacy electronically.
1. Select Patient's Pharmacy from the toolbar to document preferred patient's pharmacy.
2. If late discharge is expected, ask patient if it is a 24-hour pharmacy to prevent transferring and cancelling prescriptions.
3. Remember to document if “no pharmacy” is available.

### PACU Phase I Workflow

### Associate Monitor

1. In IView, click the Associate Monitor icon.
2. Select the monitor to associate the vitals to the patient.
3. Click Associate.

### Document Arrival Time

Open the Tracking Board and manually set event PACU In on the PACU tab.

### Review Surgical Viewpoint for Summary

Review the Perioperative tab for a summary of your patient's chart. This page will include some surgery specific information, vitals, labs, lines, orders, etc. Surgical Viewpoint will be utilized when conducting handoff reports.

### Review Orders

1. From PowerOrders, review any PowerPlans or orders present from anesthesiologist or surgeon. These will include ANES and SURG PACU PowerPlans. For other departments, the prefix may be CARD, GI, etc.
2. Initiative orders as needed.
   Remember to look in the Individual Order section for any additional orders present. SURG PostOp Surgeon PowerPlans are inpatient orders to be completed by the floor. These include the CARD or GI Postop PowerPlans if applicable to your patient. If you have any doubt, contact the physician.
   If holding a patient and an order is needed, call Registration or Bed Control to place in a virtual bed. After patient's admission is changed (which is noted in the “LOC” of the patient banner bar), initiate the PowerPlan to complete any needed orders.

### IView Documentation

1. After association, click the appropriate time to upload vitals from the Associate Monitor.
2. Confirm and accept by clicking the green check mark.
3. Document all sections pertinent to patient in the Perioperative Quickview, PACU, I&O, Perioperative Lines, Perioperative Tubes & Drains, etc. IView bands.
4. Add a Dynamic Group for any tubes, drains, or incisions not present prior to surgery, then document the assessment. Continue to document in other bands pertinent to your patient.
5. Add Navigator band if needed (e.g., Pediatric, Respiratory Therapy, etc.).
   Within IView, click View-Layout-Navigator Bands-Select Band.
   Click arrow to move to current box.
   Close patient's chart and reopen.
6. Click the green check mark to sign the documentation when purple documentation is present.
   Customize sections within IView by clicking the Customize icon and selecting the section you want to display in the band. This option will only be available for the current patient.
   Customize time display by right-clicking on time columns and selecting desired frequency.

### Specimen Collection

Specimen will be available for collection upon placing the order. If you do not collect specimen, document it in the order as “Lab Collect.” The order will be sent to the Lab to obtain the specimen.

### If the Nurse Collects the Specimen:

1. Select Specimen Collection from toolbar.
2. Scan the patient's ID band (this will pull up the patient information on the device).
3. To print additional labels (if not printed when order was placed), select Label Reprint from the toolbar.
4. Scan label on the specimen. A checkmark will appear next to the specimen name on the screen.
5. Sign the collection after all the specimens have been collected. Write initials and time collected on specimen.
7. Send to Lab.

### Medication Administration

1. Review Power Orders/MAR for medication orders.
2. Complete Nurse Review if the eyeglasses icon is present.
3. Document administration of the medications by selecting Medication Administration at the top of the screen and following prompts. If medications do not appear, change the search criteria from the date on the grey band.
4. Always scan. Report scanning issue with either Care Administration functions to Charge Nurse and IT.

### Handoff

Remember to document handoff in the Communication Section in IView. Use Surgical Viewpoint to assist in handoff communication.
Also use PowerOrders to confirm all orders that are pending/completed on the patient (especially with the Inpatients).

### Discontinue PowerPlans

Discontinue any Surgical/Procedural PowerPlans and ensure all orders have been completed. Communicate this in the handoff report. Do not discontinue floor orders.

### Finalize Chart

1. Confirm all Case Times are documented.
2. Document acuity levels accordingly.
   Diagnostic Patients=0 (Time In and Patient Ready)
   Surgical/Procedural Patients=1 (Patient In and Patient Ready)
   Holding Patients=2 (Patient Ready and PACU Discharge Time)
3. Review the Pick List and document items used. If no items are used, it must be documented as “no items used.”
4. Finalize chart by clicking the green flag. This allows for the charges to process.

### Disassociate Monitor

Click on the Association icon and disassociate the monitor from the patient.

### PACU Procedural Documentation

Cases performed in PACU (e.g., shoulder manipulations, cardioversions, etc.) must be completed in the Intraop segments.
1. Select Perioperative Documentation Intraop tab and complete documentation for the following segments:
   Case Times
   Case Attendees
   General Case Data
   Timeout
   Surgical
   Procedural
2. Select Case Logging Document from toolbar. Click to remove all sections except Timeout Document Reason.
3. Click OK.
4. Document in each of the above sections, paying close attention to the number of pages in each segment. Every grey box must have a checkmark.
5. After the five segments are complete, a green checkmark will display.
6. Complete the Pick List as “no items used” for this Intraop section.
7. Save and finalize the Intraop document.
8. If applicable, add Med Administration, Equipment, Fire Risk, or Departure from OR.
   Peripheral Nerve Blocks are not part of this documentation

### PACU Phase II Workflow

### Review Surgical Viewpoint

This is a summary page to review with the PACU/Circulator during handoff report.

### Document Arrival Time

Open the Tracking Board and manually set an event for arrival to PACU Phase II on the PACU tab.

### Review Orders

1. From PowerOrders, review any PowerPlans or orders that may have been placed in a planned state by anesthesiologist or surgeon/physician.
2. Initiate if applicable.
3. If no orders are present, notify the Charge Nurse and contact the physician for orders. Remember to check for paper orders. All paper orders must be entered into Cerner.

### Discharge Summary (Surgical Viewpoint)

Discharge PowerPlan orders will include discharge order home, follow-up instructions, and an special surgery-related instructions.
Review the Discharge Summary for the required documentation by surgeon (e.g., Discharge Diagnosis Medication Reconciliation, and Discharge Order). If not completed, contact the Charge Nurse.

### IView Documentation

1. Document all sections pertinent to patient in the Perioperative Quickview, PACU II, I&O, Perioperative Lines, Perioperative Tubes & Drains, Education, etc. IView bands.
2. Add Navigator band if needed (e.g., Pediatric, Respiratory Therapy, etc.).
   Within IView, click View-Layout-Navigator Bands-Select Band.
   Click arrow to move to current box.
   Close patient's chart and reopen.
3. Customize sections within IView. Click on Customize icon then select the section to display in the band.
4. Click the green check mark to sign the documentation when purple documentation is present.

### Medical Administration

1. Review PowerOrders/MAR for medication orders.
2. Document administration of medications by selecting Medication Administration at the top of the screen and follow prompts.
3. Always scan.

### Discharge Process

1. Click the Discharge Process icon located at the top of the Surgical Viewpoint.
2. Confirm Discharge Order is present.
3. Complete sections by clicking on the Notepad icon.
4. Review material to be printed by selecting the Patient tab in the middle of the screen.
5. After reviewing with caregiver, click the yellow highlighted box.
6. Click the Sign and Print to print the following:
   Follow-up
   Medication Leaflets
   Patient Education
   Discharge Summary
   Summary Instructions

### Finalize Chart From Preoperative Document:

1. Confirm case times are documented.
2. Review the Pick List and document items used.
3. Select either the Diagnostic or Surgical PostOp Care Pt.
4. If no items are used, document “no items used.”
5. Finalize chart by clicking the green flag. This allows for the charges to process.

### Intraop and Documentation

Intraoperative documentation is a record of case times, case attendees, operative procedure(s), case information, procedural documentation, specimens/cultures, and medications. It also involves managing segments, completing the Pick List, and finalizing the document
1. Upon receiving the assignment from the Intraoperative Tracking Board, prepare the surgery suite.
2. Review the patient chart from the or by reviewing the Surgical Viewpoint and/or Results Review.
3. Review patient information in PAT Adult and/or Preprocedure Adult (or pediatric) PowerForm. If the form has not been signed or saved, it will not be accessible through Form Browser.
4. When arriving in the Preop area, check the Tracking Board to determine the location of the patient and the name of the Preop nurse who prepared the patient for surgery.
5. Log on to SurgiNet and locate the patient's Preprocedure Adult PowerForm. Review the chart and get a brief report from the Preop nurse about the patient.
6. Facility policy will determine who completes the Out of the PreOp time segment. If you made any changes to the Preprocedure PowerForm, sign the document using the green checkmark.
7. Transport the patient to the OR suite, sign in to SurgiNet, and complete intraoperative documentation and place specimen/culture orders.
8. Document in IView any blood products given and epidural that leaves the OR with the patient.
9. Complete the Pick List.
10. Review documentation for completeness and finalize the Intraoperative record.
11. Prepare the patient for transfer and hand off to the PACU/Unit nurse.
12. Follow facility protocol for hand off to PACU/Unit.

While the above provides generic descriptions of workflows for various procedures using various software packages, it should be understood that many of the workflows can be completed with the device of the present invention, allowing users to streamline their work processes while not having to be present in front of a conventional computer terminal.

It should be understood, of course, that the foregoing relates to exemplary embodiments of the invention and that modifications may be made without departing from the spirit and scope of the invention as set forth in the following claims.

What is claimed is:

1. A method for providing and gathering information to and from medical personnel, comprising:
disposing glasses on the medical personnel, the glasses including lenses for displaying information and a wireless communication system for sending and receiving information.

2. The method of claim 1, further comprising speaking commands into a microphone disposed in the glasses, the commands operable to display menus, select items, and log medical chart information.

3. The method of claim 2, further comprising turning the microphone off when not in use to prevent inadvertent section of the commands.

4. The method of claim 1, further comprising interconnecting the glasses to a power source via a charging port in the glasses.

5. The method of claim 1, further comprising identifying a patient and accessing charts of the patient via use of a camera in the glasses.

6. The method of claim 5, wherein the camera identifies the patient by facial recognition.

7. The method of claim 5, wherein the camera identifies the patient by scanning a code associated with a room in which the patient is seen by the medical personnel, the code identifying the room and linking with a scheduling program to determine the patient scheduled in that room at that time.

8. The method of claim 1, further comprising viewing an augmented reality via a camera, disposed on the glasses, onto the lenses of the glasses.

9. A computer implemented method for providing patient care, comprising:
wearing, by medical personnel, glasses having a wireless connection configured to interface with an electronic medical record of a patient;
reading, by a camera disposed on the glasses, a patient identifier, the patient identifier identifying the patient being seen by the medical personnel; and
receiving, via the wireless connection, a medical record associated with the patient, the medical record being displayed to the medical personnel on the lenses of the glasses.

10. The method of claim 9, further comprising charging a power supply of the glasses via a charging port disposed in the glasses.

11. The method of claim 9, further comprising speaking into a microphone disposed in the glasses to access menus and specific information of the medical record.

12. The method of claim 9, further comprising illuminating a region ahead of the glasses via a light disposed on one or both sides of the glasses.

13. The method of claim 9, wherein the camera identifies the patient by facial recognition.

14. The method of claim 9, wherein the camera identifies the patient by scanning a code associated with a room in which the patient is seen by the medical personnel, the code identifying the room and linking with a scheduling program to determine the patient scheduled in that room at that time.

15. The method of claim 9, further comprising viewing an augmented reality via a camera, disposed on the glasses, onto the lenses of the glasses.

16. The method of claim 9, further comprising turning the microphone off when not in use to prevent inadvertent section of the commands.
